(19) United States
(12) Patent Application Publication
(10) Pub. No.: US 2004/0087993 A1
(43) Pub. Date: May 6, 2004

(54) Auxiliary surgery tool for protruding resected part

(76) Inventor: Shyh-Jen Wang, Taipei (TW)

Correspondence Address: Shyh-Jen Wang, P. O. Box 55-846, Taipei, 104, TW

(21) Appl. No.: 10/216,961

(22) Filed: Aug. 6, 2002

(51) Int. Cl. A61B 17/00

(52) U.S. Cl. 606/190

(57) ABSTRACT

An auxiliary surgery tool includes: a handle, a protruding portion generally formed as a conical shape on a front portion of the handle, and a probe portion formed on a front end of the protruding portion, whereby upon insertion of the probe portion into a resected organ part or canal and upon a forward thrusting of the protruding portion, the resected organ part or canal will be gradually bulged, protruded or even unwound for enhancing a smooth and safe suturing for successfully anastomosing the damaged organ part such as a resected rectal poriton.

### BACKGROUND OF THE INVENTION

In a surgical operation, the resected part should be protruded for anastomosing with an auto suture or by manual suture. For instance, a residual part of a resected rectum, after being operated for removing its tumour or damaged portion, should be protruded inwardly towards the abdomen wall to be anastomosed with the upper intestinal canal for normal fecal disposal through the rectum and anus. However, it is very difficult to perform the anastomosis in a narrow pelvic cavity for a shortened residual rectal portion which may be retracted into a deep or low portion in the pelvic cavity. Therefore, it may easily cause operation failure, leakage of gastrointestinal tract or even death due to complications.

The present inventor has found the drawbacks of the conventional surgical operation, and invented the present auxiliary tool for smoothly completing anastomoses.

### SUMMARY OF THE INVENTION

The object of the present invention is to provide an auxiliary surgery tool including: a handle, a protruding portion generally formed as a conical shape on a front portion of the handle, and a probe portion formed on a front end of the protruding portion, whereby upon insertion of the probe portion into a resected organ part or canal and upon a forward thrusting of the protruding portion, the resected organ part or canal will be gradually bulged, protruded or even unwound for enhancing a smooth and safe suturing for successfully anastomosing the damaged organ part.

### BRIEF DESCRIPTION OF THE DRAWINGS

FIG. 1 is a perspective view of the present invention.

FIG. 2 is an illustration showing the application of the present invention when used for a rectal anastomosis.

### DETAILED DESCRIPTION

As shown in the drawing figures, the auxiliary surgery tool of the present invention comprises: a handle 1, a protruding portion 2 formed on a front portion 11 of the handle 1, and a probe portion 3 formed on a front end of the protruding portion 2. The tool of the present invention may be made of plastics or alloy, but not limited in this invention.

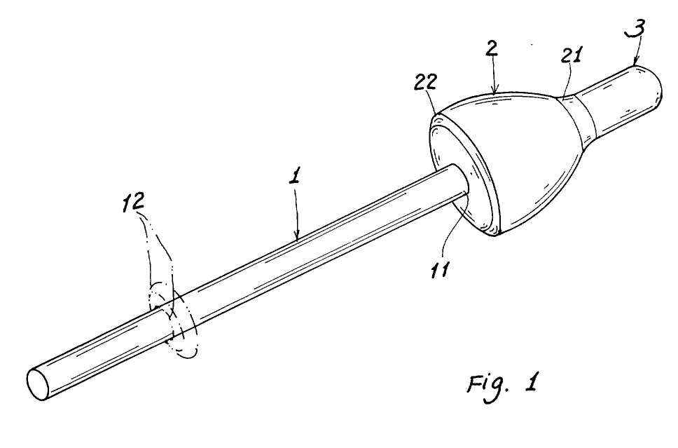

A knob portion 12 such as dotted line shown in FIG. 1 may be formed on a proper portion of the handle 1 to serve as a stopper of the handle to prevent the hand from sliding on the handle when inserting the tool into the anus A of the patient. However, the knob portion 12 may also be eliminated from the handle 1 if necessarily.

The protruding portion 2 is formed as a conical shape or truncated cone shape, having a contracted end portion 21 adjacent to the probe portion 3 and an enlarged end portion 22 opposite to the contracted end portion 21 and adjacent to the handle 1; with the enlarged end portion 22 tapered forwards towards the contracted end portion 21.

The probe portion 3 and the contracted end portion 21 each has a diameter ranging from 80˜150% of an inside diameter of a human rectum if the present invention is provided for a rectal anastomosis.

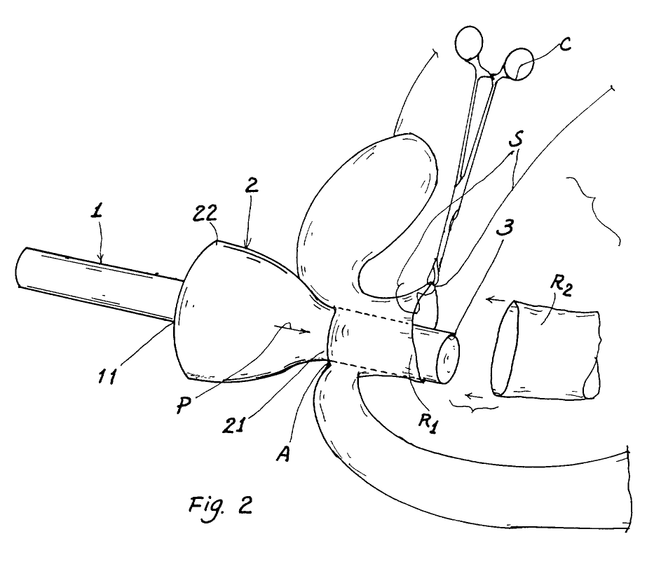

When it is used in a rectal anastomosis as shown in FIG. 2, the probe portion 3 is inserted into the patient's anus A and the protruding portion 2 is retained against a bottom portion of the patient's pelvis and then gradually urged into a residual rectal portion R1 of the patient's rectum. The protruding portion 2 of the present invention will help protrude the residual rectal resection (which is resected such as for removing a rectal tumour) inwardly towards the patient's abdomen wall (such as the arrow direction P). Accordingly, the resected rectal portion R1 will be bulged, unwound and protruded from its retracted resected state to a proper “protruding” distance (or an enough “space”) adapted for a convenient suturing such as for stitching the suture S as fastened by a clamp C as shown in FIG. 2.

Therefore, the present invention will help “protrude” a retracted resected rectal portion for an ergonomic anastomosing. Otherwise, it will be very difficult to perform a rectal anatomosis especially in a narrow space in the patient's pelvic cavity.

The protruding portion 2 is conically shaped having a conical circumferential surface tapered forwards so that the inward urging of the tool of the present invention will gradually bulge, protrude or unwind the resected residual rectal portion R1 as retracted at the anus A, without suddenly thrusting or injuring the rectum, thereby smoothening the surgical operation.

For performing purse-string sutures, the present invention is especially helpful for protruding the residual retracted resected rectal portion R1. After connecting the rectal portion R1 proximal to the anus A with the upward intestinal portion R2, the rectum will then be anatomosed.

The present invention may provide an aid for a rectal anastomosis either by manual suturing or automatic suturing.

For preventing slipping or loosening when holding the handle 1 of the present invention, the surface of the handle 1 may be finished or coated with anti-slipping surface by any conventional processing or surface treatment methods, not limited in this invention.

All elements including the handle 1, the protruding portion 2 and the probe portion 3 may be integrally formed by molding processes; or may be mechanically joined or fastened.

The probe portion 3 may be formed as a rod or a tube having a round tip front end.

The present invention may be modified without departing from the spirit and scope of the present invention.

The present invention is especially provided for rectal anastomsis. But, it may also be used to protrude any resected organ part or canal after being surgically resected in order for an ergonomic suturing.

I claim:

1. A surgery tool comprising:
a handle;
a protruding portion formed on a front portion of said handle; and
a probe portion formed on a front end portion of said protruding portion;
said protruding portion tapered forwards from said handle towards said probe portion to form a conical shape of said protruding portion; whereby upon insertion of said probe portion into a resected organ part or canal of a patient and upon inward urging of said protruding portion within the resected organ part or canal, the resected organ part or canal will be protruded, bulged or unwound for an ergonomic anastomosis thereof.

2. A surgery tool according to claim 1, wherein said handle is formed with a knob portion thereon.

3. A surgery tool according to claim 1, wherein said protruding portion on said handle includes a contracted end portion adjacent to said probe portion and an enlarged end portion opposite to said contracted end portion and adjacent to the handle, said enlarged end portion tapered forwards towards said contracted end portion to form a conical shape of said protruding portion.

4. A surgery tool according to claim 3, wherein said probe portion and said contracted end portion each has a diameter ranging from 80˜150% of an inside diameter of a human rectum, whereby upon insertion of the surgery tool into a resected rectal portion of a patient by inserting the probe portion through the patient's anus and upon inward urging of said protruding portion, the patient's resected rectal portion will be protruded, bulged or unwound for an ergonomic rectal anastomosis.

5. A surgery tool according to claim 1, wherein said handle is formed with anti-slipping surface on said handle.

6. A surgery tool according to claim 1, wherein said probe portion is formed as a rod having a round-tip front end.

7. A surgery tool according to claim 1, wherein said handle, said protruding portion and said probe portion are integrally formed.

8. A surgery tool according to claim 1, wherein said handle, said protruding portion and said probe portion are mechanically joined or fastened.

9. A surgery tool for rectal anastomosis comprising:
a handle;
a protruding portion formed on a front portion of said handle; and
a probe portion formed on a front end portion of said protruding portion;
said protruding portion tapered forwards from said handle towards said probe portion to form a conical shape of said protruding portion; whereby upon insertion of the surgery tool into a resected rectal portion of a patient by inserting the probe portion through the patient's anus and upon inward urging of said protruding portion, the patient's resected rectal portion will be protruded, bulged or unwound for an ergonomic rectal anastomosis.
